(19) United States
(12) Patent Application Publication
(10) Pub. No.: US 2003/0163856 A1
(43) Pub. Date: Aug. 28, 2003

(54) Process for generating cytoplasmic male sterile line in rice and other crops by rna editing

(76) Inventors: Villoo Morawala Patell, Bangalore (IN); Naganand Rayapuram, Bangalore (IN); Mahesh Venkataramiah, Kamataka (IN); Joy Joma, Kamataka (IN); Sulip Kumar Goswami, Andrha Pradesh (IN)

Correspondence Address: Sidley Austin Brown & Wood, 1501 K Street N W, Washington, DC, 20005, US

(21) Appl. No.: 10/204,850

(22) PCT Filed: Feb. 26, 2001
(86) PCT No.: PCT/IN01/00024

(30) Foreign Application Priority Data
Feb. 25, 2000 (IN) 153/MAS/2000

(51) Int. Cl. A01H 5/00; C12N 15/82

(52) U.S. Cl. 800/320.2; 800/287

(57) ABSTRACT

The present invention relates to a cytoplasmic male sterility (CMS) transgenic plant prepared by RNA editing for expressing unedited nad 9 gene disenabling ATP production in the mitochondria of plants and a process for preparing the same. The said process comprises: cloning an un-edited nad 9 gene by digesting the PCR product obtained from mitochondrial DNA to obtain pNG3 cloning crop with un-edited nad 9 gene with the targeting sequence under ubiquitin promoter a NOS terminator to obtain pNG11 co-bombarding pNG11 constructs in a manner as herein described with hygromycin gene (pLAU6 hph construct) to generate plants containing un-edited nad 9 gene; analysing un-edited nad 9 (pNG11) construct with a control plant having edited nad 9 gene, pNG10 to determine the presence of nad 9 gene.

### FIELD OF INVENTION

The present invention is related to Cytoplamsic male sterility (CMS) transgenic plant and a process for preparing the same using RNA editing as a molecular tool. This invention in particular relates to the development of rice hybrids (CMS) by using RNA editing.

### BACKGROUND OF THE INVENTION

CMS is a maternally inherited trait in plant mitochondria resulting in abortion of pollen grains after meiosis during microsporogenesis. The high level of interest in CMS is due to its economic importance in the production of hybrid seeds. The plants derived from cross pollination remain male sterile unless the nuclear genome of the pollen parent carries nuclear male fertility restorer (MFR) alleles (1). The ability to produce lines containing recombinant mitochondrial genomes has been extremely valuable for studying CMS. Another valuable genetic resource is a single dominant nuclear gene called Rf which reverses the CMS trait. Comparison between the fertility restored and sterile plants carrying sterile cytoplasms has yielded some important information. This can be correlated at the genetic level in some cases by the presence of chimeric genes resulting from recombination events between mtDNA (2,3) sequences and manifested biochemically by a significant decrease in ATP production. Male

The second Green Revolution is dependent on major advances in the application of molecular biology to crop plants, particularly rice. Modern molecular biology has developed a host of techniques, which will allow the analysis of crop genomes and their alteration in ways suitable for higher yields, increased disease resistance and tolerance to extremes in the environment. Rice is the second largest annual, cereal crop and grown in small land holding and manually harvested unlike wheat. The cultivated Oryza sativa, is an annual cereal crop with long leaves bearing an inflorescence (panicle) composed of spikelets with flowers producing seed or grain. It comprises approximately 25 species, domesticated in India around 3000 B.C. and later spread to China, Indo-china, Indonesia and the Japanese islands. China and India account for almost 60% of the global production, approximately 200 million tons. Rice yield depends on geographical location, weather conditions, and methods of cultivation. Rice constitutes the world's single largest market for agrochemicals consuming nearly $3 billion annually therby presenting a major opportunity for biotechnology. In India, between 1930-1980 rice production more than doubled with current figures of 420 million tons cultivated over 350 million acres. The increase was due to utilisation of improved varieties and agronomic practices.

Hybrid rice cultivation indirectly promotes cytoplasmic uniformity. In the development of new hybrids, considerable time is spent on developing the cytoplasmic male-sterile (CMS) lines. It takes 5 to 6 backcross generations to convert a line with a desirable nuclear background to a male-sterile line that can be used in hybrid seed production. The need to identify and design a quicker approach using molecular tools to generate new sources of cytoplasms is immediate.

RNA editing is a post-transcriptional process resulting in RNA altered by C to U in plant mitochondria. RNA editing changes the nucleotide sequence of an RNA molecule from that of the DNA template encoding it, thereby defying the central dogma of molecular biology (4,5). This post-transcriptional process results in RNA altered by C to U transitions and less often from T to C (6,7). Critical nucleotides in the codons are edited leading to an amino acid change, in most cases, and at times to the creation of previously non-existent initiation and termination codon to finally assemble a functional gene. After RNA editing, the sequence of the corresponding protein is different from that of the non-edited genomically predicted protein. Modifications of proteins involved in the complexes of the respiratory chain have often been shown to be responsible for male-sterile phenotype because they affect the function of complex I of mitochondrial respiratory chain. Editing events are sometimes species specific (8) and in some cases silent, partial or identical in conserved regions as in Oenothera and wheat, maize or pea (9). Editing has also been observed in the creation of splice sites in wheat and Oenothera (10,11).

The mitochondrial (mt) genome of vascular plants is characterized by a number of distinct features. These genomes are larger than non plant mitochondrial genomes and their physical structures is heterogenous due to repeats responsible for active DNA rearrangements. The complete sequencing of the mitochondiral genome of Arabidopsis thaliana (Unseld, 1997#2562) confirm that, although a large proportion of the higher plant mitochondiral DNA is noncoding, more than 50 open reading frames (orf) are present in the genome, including the normal set of genes present in the mt genome of animals but also ribosomal protein genes, new genes similar to bacterial genes involved in cytochrome c biogenesis (Gonzalesz, 1993#1874; Schuster, 1994#2194; Bonnard, 1995#2331; Gruska, 1995#2340) and genes of unknown function like orfB and orf25. The presence of chloroplastic DNA insertions has been reported in a number of different species, supplying mitochondria with several functional tRNA genes (Marechal, 1987#810). These insertions, together with the presence of introns and frequent repeated sequences contribute to the large size of higher plant mt genome.

The uniqueness of this genome is also illustrated by the mechanism required for its expression which include both cis- and trans-splicing as well as RNA editing. In most cases, mRNA editing leads to an amino acid change, including creation of previously non-existent initiation and termination codon. Nearly all transcripts of all protein coding genes are edited, resulting most often in an increase in the similarity of the protein sequences between species. A similar C to U mRNA editing activity is also found in the chloroplast of the higher plants (Maier, 1996#2560) structured RNAs, such as introns and tRNAs were also found to be edited in mitochondria of several species (Wissinger, 1992#1608; Marechal-Drouard, 1993#1991). Higher plant mitochondrial introns were first detected in the cox2 gene (Fox, 1981#367), they belong to group II introns which are characterized by their structure (Michel, 1983#1764; Michel, 1989#820) and the mechanisms employed for their removal (Cech, 1990#17645). In plant mitochondria some of this group II introns have been split by recombination, dispersing exons at different genomic locations. Therefore separated transcripts need to be connected by trans-splicing. This phenomenon, first described in nuclear genes (Borst, 1986#1778), is also required for the expression for the chloroplast rps12, (Koller, 1987#255) and psaA (Goldschmidt-Clermont, 1991#1394) genes.

The NADH-ubiquinone oxido-reductase (complex-I) comprises more than 25 protein sub-units, it is the largest of the respiratory complexes and it is located in the mitochondrial inner membrane (Herz, 1994#2080). This enzyme catalyses the oxidation of NADH and the transfer of electrons to Ubiquinone. Up to now the genes were nine mitochonrially encoded subunits have been identified in higher plant mitochondria.

nad1, nad2, nad3, nad4, nad4L, nad5 and nad6 genes (Schuster, 1994#2195) have counter parts in mammalian mitochondria in contrast to nad7 (Bonen, 1994#2123) and nad9 (Lamattina, 1993#1491) which are encoded in the nuclear genome is mammals (Walker, 1992#1834). The expression of nad genes give a good illustrations of the various range of complexities that can be found in higher plant mt genomes. nad3, nad4L, nad6 and nad9 are continuous genes while nad4 (Lamattina, 1991#1080) and nad7 (Bonen, 1994#2123) are interrupted by several cis-splicing introns. The genes for nad1 (Wissinger, 1991#1124; Chapdelaine, 1991#1097), nad2 (Binder, 1992#1416) and nad5 (Knoop, 1991#1250; Pereira de Souza, 1991#1128) are cis-spliced and trans-spliced in various plant species showing the most complex expression patterns.

The structure and expression of wheat nad2, the mitochondrial gene coding for subunit2 of the NADH-ubiquinone reductase whose expression exemplifies some characteristics of the vascular plant mitochondrial genome, Cis-, trans-splicing and mRNA editing are necessary for the correct expression of this gene, has been studied. In wheat mitochondria, the gene coding for subunit2 of the NADH-ubiquinone Oxido-reductase (nad2) is divided in five exons located in two distinct genomic regions. The first two exons of the genes, a and b, are transcribed 22 kb downstream of exons c, d, and e, from the same DNA strand. All introns of nad2 are of group II. Trans-splicing of the exons b and c has to be postulated to bring together the two independently transcribed coding sequences. DNA rearrangements with in domain IV loop appear to be responsible for this gene organization since the trans-splicing event involves base pairing of the two precusor RNAs in the stem of the interrupted domain IV. A gene coding for tRNATyr is located upstream of exon c and may be co-transcribed with exons c-e. In addition to splicing processes, mRNA editing is also required for the correct expression of nad2. The mature mRNA is edited at 36 position, randomly distributed over its five exons resulting in 28 codon modifications. Editing increases protein hydrophobicity and conservation

A very narrow cytoplasmic base can result in a crop being vulnerable to disease, pest outbreaks and adverse environmental conditions. Most of the present day hybrids have a very narrow cytoplasmic base like the “WA” cytoplasm rendering the crop vulnerable to disease and pest attacks and adverse environmental conditions.

present day hybrids have a very narrow cytoplasmic base like the “WA” cytoplasm rendering the crop vulnerable to disease and pest attacks and adverse environmental conditions.

It is therefore the principal object of the present invention to identify and develop new sources of CMS conferring cytoplasm.

Yet another object of the present invention is to optimize the use of genetically engineering over-expression of an unedited version to generate CMS and an anti-sense version of the same for restoration of the nuclear fertility.

The objective of this invention is also to combine the fundamental importance of RNA editing in accurate transcript processing and with a more applied approach to generate CMS lines in crop plants, in particular rice.

Still further object of this invention is to combine the fundamental importance of RNA editing in accurate transcript processing and with more applied approach to generate CMS lines in crop plants, in particular rice.

Yet another object of this invention is to correlate the expression of the unedited version of the gene with the emergence of male sterility. The male-sterile phenotype is the consequence of mitochondrial dysfunction affecting normal anther development and reducing the formation of pollen grains.

To achieve the said object the present invention relates to a cytoplasmic male sterile (CMS) transgenic Indica rice, Basmati 370 variety of commercial importance, obtained by RNA editing by expressing the unedited nad9 gene thereby causing an alteration in the functioning of the proton pumping multi protein complexes of the mitochondrial inner membrane resulting in an impairment of the nad9 subunit alignment, and, loss of function of the complex I (NADH-ubiquionone oxido-reductase), further resulting in a decrease in the cellular energy levels, causing total pollen abortion, thus, giving rise to a 100% male sterile rice plant.

Said cytoplasmic male sterile (CMS) transgenic Indica rice plant is selected from the group of Oryza sativa (rice), wheat (Triticum aestivum), corn (Zea Mays), soyabean (Glycine max) and the like.

Said cytoplasmic male sterile (CMS) transgenic Indica rice plant is Oryza sativa (rice) plant.

The present invention also relates a process for preparing a cytoplasmic male sterile (CMS) transgenic Indica rice plant, comprising the steps of:

i. inserting the nucleic acid sequence (unedited nad9 gene) capable of modifying male sterility in plants and is associated with a sequence Arabidopsis thaliana cDNA (At-mRBP1A) capable of transferring the protein expressed by said coding region to the mitochondria,

ii. a subunit (nad9) of Complex I (NADH-ubiquionone oxido-reductase) of the respiratory gene, leading to a reduction of ubiquinone by an electron transporter in the mitochondrial inner membrane, said Complex I occupying a strategic position in the electron flow and mutations perturbing its assembly to induce male sterility,

iii. in Complex I, nad9 is situated in the iron sulphur subfraction facing the mitochondrial matrix and its deduced amino acid composition indicating an hydrophilic protein edited in 12 positions, thereby, the unedited nad9 gene leading to an alteration in the functioning of the proton pumping multi protein complexes of the mitochondrial inner membrane resulting in an impainnent of the nad9 subunit alignment, and, loss of function of the complex I, further resulting in a decrease in the cellular energy levels, causing total pollen abortion, thus, giving rise to a 100% male sterile rice plant,

iv. regenerating and culturing said transgenic plants and measuring the production and the viability of pollen from said transgenic plants.

Restoration of male fertility to transgenic male sterile plants is carried out by introducing at least one copy of an anti sense (unedited nad9) nucleic acid sequence, targeting into the mitochondria by Arabidopsis thaliana cDNA (At-mRBP1A) targeting peptide, as a result of which transgenic male fertile plants can be obtained.

A novel mitochondrial targeting peptide is cloned by means of PCR by digesting at the Sac1 and the Xba1 sites from Arabidopsis thaliana cDNA (At-mRBP1A) in pBSK vector and named, pNG1.

The edited nad9 gene is obtained by RT-PCR from the rice plant (IR64 variety), the PCR product is cloned into pNG1 by digesting at the Xba1 and the BamH1 sites to obtain a construct, pNG2.

The unedited nad9 gene obtained from the rice plant (IR64 variety) mitochondrial DNA was cloned into pNG1 by digesting at the Xba1 and the BamH1 sites to obtain a construct, pNG3.

The edited nad9 gene together with the targeting sequence is cloned under the ubiquitin promoter and the NOS terminator to obtain a construct pNG10.

The unedited nad9 gene along with the targeting sequence is cloned under the ubiquitin promoter and the NOS terminator to obtain a construct pNG11.

The pNG10 construct is co-bombarded with the hygromycin gene (pLAU6 hph construct) to generate edited nad9 Basmati 370 rice plants.

The pNG11 construct is co-bombarded with the hygromycin gene (pLAU6 hph construct) to generate unedited nad9 Basmati 370 rice plants.

The editid nad9 Basmati 370 fertile transgenic rice plants are generated.

The unedited nad9 Basmati 370 transgenic rice plants showed pollen abortion resulting in 100% male sterility.

The pNG11 construct displays a similar function when applied to all other crops.

The present further provides a method for preparing a cytoplamsic male sterility (CMS) transgenic plant by RNA editing for expressing unedited nad 9 gene disenabling ATP production in the mitochondria of plants, comprising the following steps:

cloning the mitochondrial targeting peptide by PCR from Arabidopsis thaliana cDNA (At-mRBP1a),

digesting the PCR product at the SacI anc XbaI sites with SacI and XbaI to facilitate cloning it into pBSK vector to obtain a construct called pNG1,

cloning and non-edited nad 9 gene obtained from mitochondrial DNA into pNG1 from the crop variety by digesting the PCR product,

digesting the cloned product having having un-edited nad 9 gene to obtain pNG3

cloning crop with non-edited nad 9 with the targeting sequence was under ubiquitin promoter and NOS terminator to obtain pNG11,

co-bombarding pNG 11 constructs with hygromycin gene (pLAU6 hph construct) to generate obtain unedited nad 9 plants,

### DETAILED DESCRIPTION OF THE INVENTION

The aim of this invention is to alter the functioning of one or more of the four proton pumping multiprotein complexes of the mitochondrial inner membrane (NADH-reductase, cytochrome c reductase, and ctochrome c oxidase or the ATP synthase complex) by using two forms of the same gene, resulting in a decrease in cellular energy levels One form would be the naturally occurring, functionally edited transcript and the other a non edited transcript resulting in a different RNA and putative protein and thus impaired subunit alignment and loss of function of the complex.

A subunit (nad9) of Complex I (NADH-ubiquinone oxido-reductase) is the first complex of the respiratory chain, leading to the reduction of ubiquinone, an electron transporter in the mitochondrial inner membrane. Complex I occupies a strategic position in the electron flow & mutations that perturb its assembly have been shown to induce male sterility. This subunit (nad9) corresponds to the 30 Kda protein of the complex I of mammals. In Complex I, nad9 is situated in the iron sulphur sub-fraction, facing the mitochondrial matrix. Its deduced amino acid composition indicates that it is an hydrophilic protein. nad9 would be the best candidate for the purpose as it is a small transcript, edited in 14 positions in wheat leading to 11 amino acid modifications, in every case resulting in a better conserved protein.

In particular, the process involved:

### Step 1

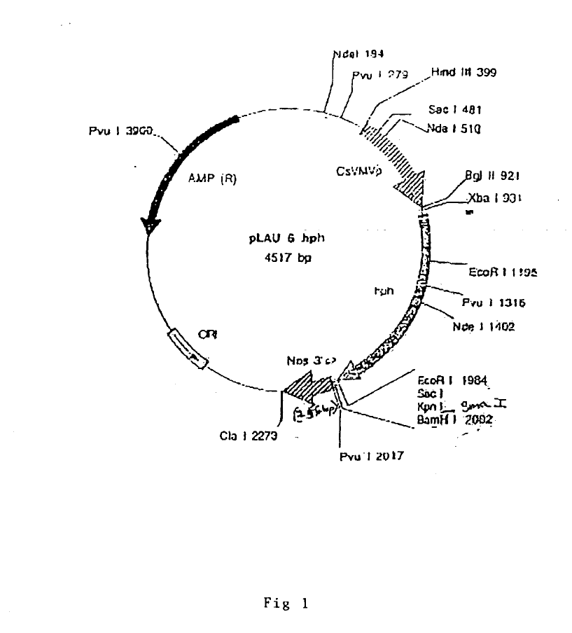

Cloning of the mitochondrial targeting sequence (FIG. 1):

The mitochondrial targeting sequence was cloned by PCR from Arabidopsis thaliana cDNA (At-mRBP1a)

Oligos:

Forward primer: aagagcTccc ATG GTC TTC TGT AAC AAA CTC G

Reverse primer: Aa tct Aga CTT GGT AGA CAT CAA CCG G

The forward primer has SacI site and the reverse primer has XbaI site to facilitate cloning in pBS(SK)

The PCR Conditions for cloning the mitochondrial targeting sequence are:

50 ng of vector harbouring the At-mRBP1a cDNA

10 ul 10×Pfu cloned buffer

2 ul 10 mM dNTP's

1 ul oligo forward (50 pmoles)

1 ul oligo reverse (50 pmoles)

1 ul of pfu polymerase

water to final volume of 100 ul

92'C 3 min

92'C 45 sec

40'C 1 min (5 cycles)

72'C 1 min

92'C 45 sec

47'C 1 min (30 cycles)

72'C 1 min

72'C 10 min

The PCR product was digested with SacI & Xba I and cloned into pBS(SK).This construct is called pNG1

### Step 2

Cloning of the rice edited nad9:

The edited nad9 is obtained by RTPCR from rice total RNA. Indica rice (IR64) seeds were germinated on moist vermiculite at 250 c in dark & allowed to grow for three weeks and the three week old seedling were used for total RNA extraction & for isolation of mitochondria

Extraction of total RNA

Grind 3 gm of tissue in liquid nitrogen, transfer to a falcon tube.

Add 9 ml of extraction buffer, vortex

Add 0.6 ml of 3M sodium acetate pH 4.8, 3.0 ml of water saturated phenol and 1.8 ml of chloroform:isoamyl alcohol (24:1), vortex

Incubate on ice for 15 min

Centrifuge for 30 min at 15000 rpm in a JA 25.50 rotar

Extract with phenol:chloroform till the inter phase is clear

To the aqueous phase add equal volume of isopropanol

Store at −20'C for 1 hr

Centrifuge at 15000 rpm for 30 min

Wash with 70% alcohol

Dry the pellet

Dissolve in water

Extraction buffer

4M Guanidine thiocyanate

25 mM sodium citrate pH 7.0

0.5% sarcosyl

0.1 mM /3-mercaptoethanol

3M sodium acetate pH 4.8

Water equlibriated phenol

Chloroform:isoamylalcohol (24:1)

Isopropanol & 70% alcohol

RT-PCR

Reverse transcription

If RNA is in ethanol, add 1/10^{th} the volume of 3M Sodium acetate, precipitate RNA for 1 hr at −20'C, centrifuge for 30 min at 12000 rpm, wash pellet with 70% ethanol, dissolve in milli Q water

Further add 500 ng of random hexamers, Incubate at 65'C for 5 min and place on ice. Further add the following:

10 ul 5×RT buffer

5 ul DTT (0.1M)

10 ul dNTP's (2 mM)

1 ul Reverse Transcriptase M-MLV (=200 units)

Then further incubate at 37'C for 2 hr

Add 5 ul ATP 10 mM

1 ul T4 polynucleotide kinase (10 units)

Incubate at 37'C for 15 min

Add 2 ul T4 DNA ligase (2 units)

Incubate at 37'C for 45 min

Oligos:

Forward: aa Tct aga ATG GAT AAC CAA TTC ATT TTC CAA

Reverse: aag gAt cCG GAA TTA TCC GTC GCT ACG

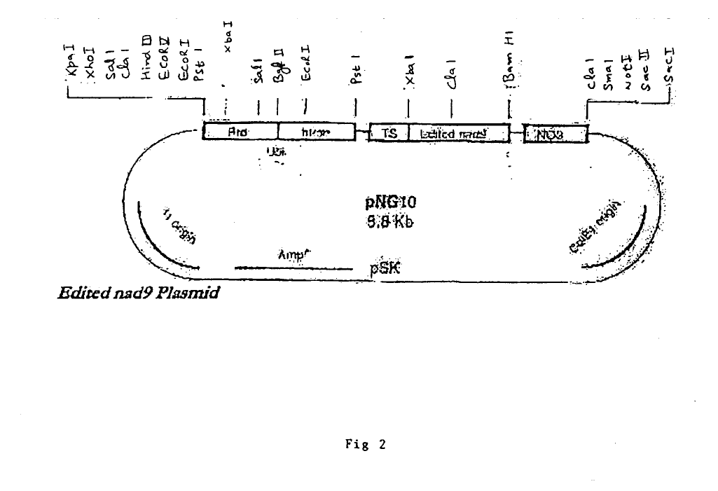

The forward primer has XbaI site and the reverse primer has BamHI for cloning edited nad 9 gene adjacent to the mitochondrial targeting sequence. PCR was carried out, according to standard conditions. The PCR product was digested with XbaI and BamHI & cloned into pNG1 and called as pNG2 From this pNG2 the edited nad9 gene was digested with Xba and BamHI and cloned into ubiquitin promoter and called as pNG 10 as shown in FIG. 2

### Step 3

Cloning of rice unedited nad9

Isolation Of mitochondria was carried out using standard protocol.

Extraction Buffer

0.4 M sucrose

50.0 mM tris HCl pH 7.5

3.0 mM EDTA

0.1% BSA

4.0 mMb -/3-mercaptoethanol

2 mM DTT

Wash Buffer-Extraction buffer without BSA & DTT

2× Gradient Buffer

0.5M sucrose

100 mM tris HCl pH 7.5

6.0 mM EDTA

Percoll gradient prepared in 2× gradient Buffer

Lysis of mitochondria to obtain mitochondrial DNA:

Approximately 75 ug of rice mitochondria were resuspended in resuspension buffer. To the resuspended mitochondria ¼^{th} volume of lysis buffer was added. The tube was inverted & phenol was added immediately followed by chloroform. Then three phenol chloroform extractions were carried out followed by chloroform extraction. To the aqueous phase 2.5 volumes of ethanol and 1/10^{th} volume of 3.0M Na acetate was added. Stored at −20'C for 30 min, spun at 13000 rpm for 20 min, washed with 70% ethanol, dried and dissolved in water

Oligos

Forward: aa Tct aga ATG GAT AAC CAA TCC ATT TTC CAA

Reverse: aag gAt cCG GGA TTA TCC GTC GCT ACG

The forward primer has Xba I site and reverse primer has Bam H1 for cloning non-adited nad 9 gene adjacent to the mitochondrial targeting sequence.

PCR

1 ul of 1:10 diluted mitochondiral DNA was taken for PCR

10 ul 10×Pfu cloned buffer

2 ul 10 mM dNTP's

1 ul oligo forward (50 pmoles)

1 ul oligo reverse (50 pmoles)

1 ul of pfu polymerase

water to final volume of 100 ul

92'C 3 min

92'C 45 sec

42'C 1 min (5 cycles)

72'C 1 min

92'C 45 sec

49'C 1 min (30 cycles)

72'C 1 min

72'C 10 min

The PCR product was digested with XbaI and BamHI and cloned into pNG1 and called pNG3.

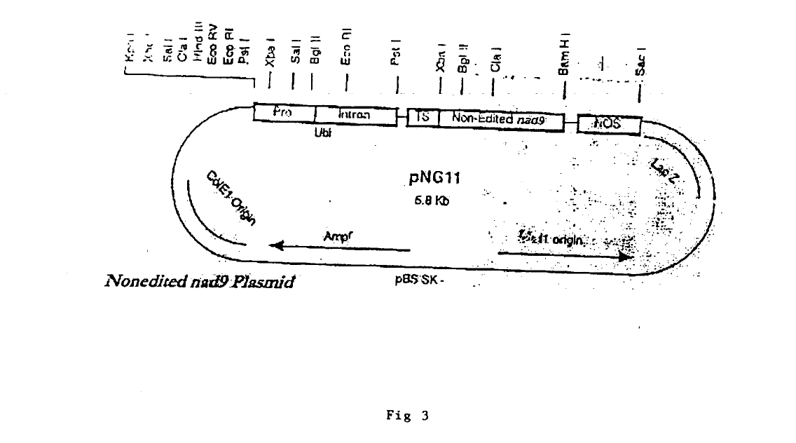

From this pNG3 the un-edited nad9 gene was digested with Xba and BamHI and cloned into ubiquitin promoter and called as pNG 11 as shown in FIG. 3

### Step 4

The edited nad9 (pNG10) and un-edited nad9 (pNG11) were used for biolistic transformation. The method of transformation is co-bombardment and so the constructs for biolistic transformation will have the gene of interest on one plasmid (pAHC27 with Ubiquitin promoter and Nos terminator and pLAU6 (from ILTAB which has the CvMV promoter and NOS terminator)) and the selection marker on another plasmid.

### EXAMPLE

Collection of plant materials: Seeds of Basmathi 370 & swarna were provided by Directorate Of Rice Research (DRR, Hyderabad, India.)

Media: MS medium (Murashige & skoog, 1962) is composed of Mssalts (MS-Macronutrients, MS micronutrients), FeEDTA, 0.5 mg/L Pyrodoxine, 1.0 mg/L Thiamine, 0.5 mg/L Nicotinic acid, 30 g/L sucrose, 2.6 g/L phytagel.

MSO medium is MS medium supplemented with 30 g/L mannitol & 30 g/L sorbitol.

RC Medium for first selection supplemented with 30 mg/L hygromycin CC medium for second selection with 50 mg/L hygromycin, 300 mg/L casein hydrolysate, 500 mg/L proline.

Pre regeneration medium with 30 g/L maltose, 50 mg/L hygromycin, 2 mg/L kinetin, 1 mg/L naphthylene acetic acid, 5 mg/L abscisic acid.

Regeneration medium with 30 g/L maltose, 50 mg/L hygromycin, 2.5 mg/L kinetin, 0.1 mg/L Naphthylene acetic acid.

½ MS medium for rooting containing ½ MS salts, ½ B5 vitamins, 10 mg/L sucrose.

Callus induction & selection of regenerable calli: De-husked mature seeds were surface sterilized in 70% ethanol for 2 min followed by 50% commercial bleach for 30 min. The seeds were then rinsed with sterile water. The seeds were then placed on petriplates containing MS media & incubated at 25 c for 14 days in dark. The primary calli induced from scutellar region were removed & subcultured on fresh MS media for a week at the same conditions. After the subculture many loosely attached small globular calli appeared on top of each compact primary callus, which were gently removed & placed on fresh MS medium. Calli of 1-3 mm in diameter were used for transformation.

Preparation of subcultured calli for bombardment: About 60 embryogenic calli, 2-3 mm in diameter were placed at the center of a petriplate containing osmoticum medium. After 4 hrs incubation on this medium the calli were immediately subjected to microprojectile bombardment using the particle accelerator, PDS-1000/He.

Microprojectile-Mediated transformation: The biolistic gun used is the PDS-1000/He gun (Bio-Rad Laboratories, USA). The size of gold used was between 1.5-3.0μ. The rupture disc pressure was 1100 psi while the helium pressure had to be 1200 psi. A vacuum of 25 mg/Hg was created in the gun chamber. The concentration of the DNA used was 5 μg/μl.

Preparation of gold suspension: 6 mg of gold particles were weighed to which 100 μl of 100% ethanol was added and was vortexed for a minute. This was centrifuged for 10 seconds at 10000 rpm. The supernatant was pipetted out and 100 μl of sterile distilled water was added to the pellet. It was vortexed and centrifuged and the same procedure was repeated. 50 μl final gold suspension was used for bombardment.

Particle coating protocol: To 50 μl of the gold suspension 5 μg of DNA was added and mixed well. To this 20 μl of 0.1M Spermidine (Sigma, Aldrich) was added and mixed at low speed on the vortex. 50 μl of 2.5M CaCl2 was added and mixed well. The mix was left at room temperature for 10 minutes. It was centrifuged for 10 seconds at 10000 rpm and the supernatant was pipetted out and 50 μl of 100% ethanol was added to the pellet. 10 μl of the sample was used to coat on macrocarrier, the membrane was allowed to dry & then used for transformation. After the transformation the calli were kept on the same plates & incubated in dark for 16 hrs.

Growth & Selection of Bombarded Cells: After 16 hrs the calli were transferred to RC 30 medium for selection & incubated at 25'c in dark for 21 days. The resistant calli were transferred to CC50 medium & incubated in dark for 18 days. Only resistant calli were transferred on to pre-regeneration media for a week. The proliferating calli were kept on regeneration media under a photo period of 16 hrs light & 8 hrs dark at 25'c. As plant lets were regenerated they were transferred into test tubes containing ½ MS medium.

Analyses of transformants will be multiple ways:

1. The presence of the transgene will be tested by Southern hybridisation with adequate probes (cDNA clones, oligonucleotides). The expression of the gene into RNA will be analysed by Northern blots and the sequence of the mRNA will be subsequently obtained after RT-PCR.

2. The importation of the protein will be followed using antisera against nad9 which are monospecific.

3. The integration of the protein in the complex I and the estimation of the damages caused by the putative mis-assembly will be assessed using a new technology of immunoaffinity.

4. Histological analysis of anthers will be carried out using toluidene blue for ultrastructural analysis.

5. Basic breeding studies on number of tillers, seed, plant length to assess the yield will be carried out.

6. Crosses will be made between edited and unedited nad9 transformed rice plants to assess for fertility restoration.

7. Pollen viability will be evaluated by the ability of pollen to germinate on specified medium.

We claim:

1. A cytoplasmic male sterile (CMS) transgenic Indica rice, Basmati 370 variety of commercial importance, obtained by RNA editing by expressing the unedited nad9 gene thereby causing an alteration in the functioning of the proton pumping multi protein complexes of the mitochondrial inner membrane resulting in an impairment of the nad9 subunit alignment, and, loss of function of the complex I (NADH-ubiquionone oxido-reductase), further resulting in a decrease in the cellular energy levels, causing total pollen abortion, thus, giving rise to a 100% male sterile rice plant.

2. A cytoplasmic male sterile (CMS) transgenic Indica rice plant as claimed in claim 1 wherein said plant is selected from the group of Oryza sativa (rice), wheat (Triticum aestivum), corn (Zea Mays), soyabean (Glycine max) and the like.

3 A cytoplasmic male sterile (CMS) transgenic Indica rice plant as claimed in claim 1 wherein said plant is Oryza sativa (rice) plant.

4. A process for preparing a cytoplasmic male sterile (CMS) transgenic Indica rice plant, comprising the steps of:
i. inserting the nucleic acid sequence (unedited nad9 gene) capable of modifying male sterility in plants and is associated with a sequence Arabidopsis thaliana cDNA (At-mRBP1A) capable of transferring the protein expressed by said coding region to the mitochondria,
ii. a subunit (nad9) of Complex I (NADH-ubiquionone oxido-reductase) of the respiratory gene, leading to a reduction of ubiquinone by an electron transporter in the mitochondrial inner membrane, said Complex I occupying a strategic position in the electron flow and mutations perturbing its assembly to induce male sterility,
iii. in Complex 1, nad9 is situated in the iron sulphur subfraction facing the mitochondrial matrix and its deduced amino acid composition indicating an hydrophilic protein edited in 12 positions, thereby, the unedited nad9 gene leading to an alteration in the functioning of the proton pumping multi protein complexes of the mitochondrial inner membrane resulting in an impairment of the nad9 subunit alignment, and, loss of function of the complex I, further resulting in a decrease in the cellular energy levels, causing total pollen abortion, thus, giving rise to a 100% male sterile rice plant,
iv. regenerating and culturing said transgenic plants and measuring the production and the viability of pollen from said transgenic plants.

5. A process as claimed in claim 4, wherein restoring male fertility to transgenic male sterile plants is carried out by introducing at least one copy of an anti sense (unedited nad9) nucleic acid sequence, targeting into the mitochondria by Arabidopsis thaliana cDNA (At-mRBP1A) targeting peptide, as a result of which transgenic male fertile plants can be obtained.

6. A process as claimed in claim 5, wherein a novel mitochondrial targeting peptide is cloned by means of PCR by digesting at the Sac1 and the Xba1 sites from Arabidopsis thaliana cDNA (At-mRBP1A) in pBSK vector and named, pNG1.

7. A process as claimed in claims 4, wherein the edited nad9 gene is obtained by RT-PCR from the rice plant (IR64 variety), the PCR product is cloned into pNG1 by digesting at the Xba1 and the BamH1 sites to obtain a construct, pNG2.

8. A process as claimed in claim 4, wherein the unedited nad9 gene obtained from the rice plant (IR64 variety) mitochondrial DNA was cloned into pNG1 by digesting at the Xba1 and the BamH1 sites to obtain a construct, pNG3.

9. A process as claimed in claim 7 wherein the edited nad9 gene together with the targeting sequence is cloned under the ubiquitin promoter and the NOS terminator to obtain a construct pNG10.

10. A process as claimed in claim 8 wherein the unedited nad9 gene along with the targeting sequence is cloned under the ubiquitin promoter and the NOS terminator to obtain a construct pNG11.

11. A process as claimed in claim 9 wherein the pNG10 construct is co-bombarded with the hygromycin gene (pLAU6 hph construct) to generate edited nad9 Basmati 370 rice plants.

12. A process as claimed in claim 10 wherein the pNG11 construct is co-bombarded with the hygromycin gene (pLAU6 hph construct) to generate unedited nad9 Basmati 370 rice plants.

13. A process as claimed in claim 11 wherein the edited nad9 Basmati 370 fertile transgenic rice plants are generated.

14. A process as claimed in claim 12 wherein the unedited nad9 Basmati 370 transgenic rice plants showed pollen abortion resulting in 100% male sterility.

15. A process as claimed in claim 14 wherein the pNG11 construct displays a similar function when applied to all other crops.
